(19) United States
(12) Patent Application Publication
(10) Pub. No.: US 2010/0256481 A1
(43) Pub. Date: Oct. 7, 2010

(54) Method and Apparatus for Providing a Wireless Multiple-Frequency MR Coil

(76) Inventors: Thomas H. Mareci, Gainesville, FL (US); Rizwan Bashirullah, Gainesville, FL (US); Brian S. Letzen, Coral Springs, FL (US); Barbara L. Beck, Micanopy, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/680,663

(22) PCT Filed: Sep. 29, 2008
(86) PCT No.: PCT/US08/78170

§ 371 Date: Mar. 29, 2010

Related U.S. Application Data
(60) Provisional application No. 60/975,721, filed on Sep. 27, 2007.

(51) Int. Cl. A61B 5/055

(52) U.S. Cl. 600/423

(57) ABSTRACT

Embodiments of the invention pertain to a method and apparatus for magnetic resonance imaging and spectroscopy (MRI/S). In a specific embodiment, the method and apparatus for MRI/S can be applied at two or more resonant frequencies utilizing a wireless RF receiving coil. In an embodiment, the wireless coil, which can be referred to as the implant coil, can be incorporated into an implantable structure. The implantable structure can then be implanted in a living body. The wireless RF receiving coil can be inductively coupled to another RF coil, which can be referred to as an external coil, for receiving the signal from the wireless implant RF coil. In an embodiment, the implantable structure can be a capsule compatible with implantation in a living body. The implantable structure can incorporate a mechanism for adjusting the impedance of the implant coil so as to alter the resonance frequency of the implant coil. In a specific embodiment, the mechanism for adjusting the impedance of the implant coil can allow the implant coil to receive at least two resonance frequencies. In an embodiment, the implant coil can receive three resonance frequencies and in a further embodiment, the implant coil can receive any number resonance frequencies. These resonance frequencies can be controlled by adjusting the impedance of the implant coil. In an embodiment, the resonance frequencies of the implant coil are selected to correlate to MRI/S signals received from living tissues.

### BACKGROUND OF INVENTION

Magnetic Resonance Imaging and Spectroscopy (MRI/S) techniques are routinely used for in vivo and in vitro studies to assess and monitor biological systems. An RF antenna, or surface coil, acts as a transducer to sense the electromagnetic energy excited in the biological system. The MRI/S experiment is an inherently low sensitivity measurement and although these techniques demonstrate excellent potential, their limited sensitivity hinders a complete characterization of some biological systems, such as deep tissue organs.

Of the total U.S. population, 7% have diabetes, and 5-10% fall under the category of Type-I diabetes, a pancreatic disorder in which insulin production is hindered, resulting in an unbalanced content of glucose in the bloodstream. Currently, there is no cure for this disease. Although daily insulin injections give people a near normal life, they are still greatly affected by a changed lifestyle and can only delay the major health consequences induced by diabetes. An alternative solution to alleviate the burden of the current treatment is the development of a tissue engineered pancreatic substitute (work currently being done in the University of Florida College of Medicine). These substitutes, call tissue constructs, would free patients from the daily insulin injections and the constant monitoring of their blood glucose levels. Central to this research is the need for an in vivo method to monitor a host's glucose regulation resulting from the tissue engineered pancreas. This will be carried out through the use of nuclear magnetic resonance (NMR) techniques to monitor tissue construct function and post-implantation physiological effects. Static multiple-resonant coils have yielded a low quality factor. A complication arises since 2-4×10⁶ cells/ml alginate are necessary to sustain sufficient oxygenation at the construct's center so that insulin secretion remains unaffected. This number of cells can barely be detected in vitro by a moderately high field NMR instrument/surface coil configuration. Evaluating such organ substitutes would greatly benefit from an MRI/S coil with high sensitivity at multiple MR frequencies of interest, allowing for a complete characterization of function and viability of the tissue. Several other examples of translational work utilize a bioartificial device, such as the kidney, liver, and lung. Circe Biomedical, Inc. (Lexington, Mass.) has developed the Bioartificial Liver HepatAssist device to replace metabolic function in patients with a failing liver. Another bioartificial liver has been developed by VitaGen, Inc. (La Jolla, Calif.), known as the Extracorporeal Liver Assist Device, and has completed Phase 1 clinical trials. These are just a few current examples of the great potential that exists for organ development. As stated in the recent issue of BBC Health, “The worldwide organ shortage means medical researchers are looking at alternative solutions”.

To uphold the quality and integrity of data submitted to the FDA and provide for the protection of human subjects in clinical trials, the FDA amended the Federal Food, Drug, and Cosmetic Act (FD&C Act) to include a clause that requires pharmaceutical companies to focus on preclinical studies on animals. When testing a new drug, NMR spectroscopic information is useful in analyzing the efficacy and safety of the drug in question. Being able to gather all of this information in one exam without multiple NMR coil changes increases the productivity of the experiments and minimizes hardship to the animal, and would significantly reduce the number of animals required, in support of recent imperatives established by the FDA. Beyond testing of animals would follow clinical trials of drugs involving human subjects.

### BRIEF SUMMARY

Embodiments of the invention pertain to a method and apparatus for magnetic resonance imaging and spectroscopy (MRI/S). In a specific embodiment, the method and apparatus for MRI/S can be applied at two or more resonant frequencies utilizing a wireless RF receiving coil. In an embodiment, the wireless coil, which can be referred to as the implant coil, can be incorporated into an implantable structure. The implantable structure can then be implanted in a living body. The wireless RF receiving coil can be inductively coupled to another RF coil, which can be referred to as an external coil, for receiving the signal from the wireless implant RF coil. In an embodiment, the implantable structure can be a capsule compatible with implantation in a living body. The implantable structure can incorporate a mechanism for adjusting the impedance of the implant coil so as to alter the resonance frequency of the implant coil. In a specific embodiment, the mechanism for adjusting the impedance of the implant coil can allow the implant coil to receive at least two resonance frequencies. In an embodiment, the implant coil can receive three resonance frequencies and in a further embodiment, the implant coil can receive any number resonance frequencies. These resonance frequencies can be controlled by adjusting the impedance of the implant coil. In an embodiment, the resonance frequencies of the implant coil are selected to correlate to MRI/S signals received from living tissues.

In an embodiment, the implantable structure can incorporate a microcontroller that can be wirelessly communicated with to instruct the microcontroller as to what resonance frequency to set the implant coil to and the microcontroller can control a mechanism for adjusting the impedance of the implant coil. In an embodiment, the microcontroller controls a varactor array to control the impedance of the implant coil. In a specific embodiment, the implant coil can be used to receive communication signals for communicating with the microcontroller. In alternative embodiments, a communication coil can be utilized in the implantable structure for receiving communication signals for providing input to the microcontroller.

The implantable structure is then implanted in living tissue such as a human being or animal. The implantation can be at a known orientation to allow interpretation of the MRI/S signal. Alternatively, the implantation structure can incorporate a mechanism for determining the orientation of the implant coil such as one or more fiducial markers visible under MRI/S or other techniques known in the art. This allows a determination to be made as to the relative orientation of the implant coil and external coil and/or the orientation of the implant coil in the magnetic field of the MR scanner, in order to enhance the coupling between the implant coil and external coil and the SNR of the MRI/S signal, respectively.

The external coil can act as the transmit coil for the MRI/S scanning or a separate transmit coil can be used.

Embodiments of the invention can allow a single coil to image tissue proximate the coil at two, three, four, or more resonance frequencies by remotely adjusting the impedance of the implant coil. The embodiment can allow optimization of the impedance matching under condition of extreme loading, such as a very small or very large sample (or patient).

### BRIEF DESCRIPTION OF DRAWINGS

FIG. 1 shows a simplified block diagram of an embodiment of the invention.

FIG. 2 shows an embodiment of a varactor/capacitor array with PIN diode switches that can be utilized in accordance with the invention.

FIG. 3 shows an overall digital system level design utilizing a microcontroller and a DAC connected to the array, in accordance with an embodiment of the invention.

FIG. 4 shows a diagram of an embodiment of a remotely-tuned, multiple frequency implantable coil system, with a detailed breakout of the 3×3×0.5 mm³ integrated chip.

FIG. 5 shows an embodiment of a microcontroller-driven varactor array with assembled modules in accordance with the invention.

FIGS. 6A-6D shows 11.1 T MR frequencies, where FIG. 6A shows ¹H: 470 MHz, FIG. 6B shows ¹⁹F: 442 MHz, FIG. 6C shows ³¹P: 190 MHz, and FIG. 6D shows ¹³C: 118 MHz.

FIG. 7 shows a mixed-signal integrated circuit fabricated in standard CMOS technology to selectively tune an implantable coil to the multiple MRI/S resonances.

FIGS. 8A-8C show a configuration of inductively coupled coils, in which the internal coil is resonant and the external coil may or may not be resonant.

FIG. 9 shows a system level block diagram of an embodiment of the subject device.

FIG. 10 shows measured performance of a wireless data link and power interface with battery charger: (a) ASK detector (b) clock and data recovery; (c) load response of regulated supply and (c) battery control loop charging.

FIG. 11 shows two loss mechanisms that arise from replacing a fixed value capacitor with a D-cap array.

FIG. 12 shows an overall architecture of a microchip in accordance with an embodiment of the invention.

FIG. 13 shows an embodiment having a microcontroller, register bank, serial interface and digitally controlled capacitor array to tune the coil.

FIG. 14 shows the required battery capacity and estimated device duration with and without a battery management system.

FIG. 15 shows a block diagram of an embodiment of the external coil with an automated impedance matching system.

### DETAILED DISCLOSURE

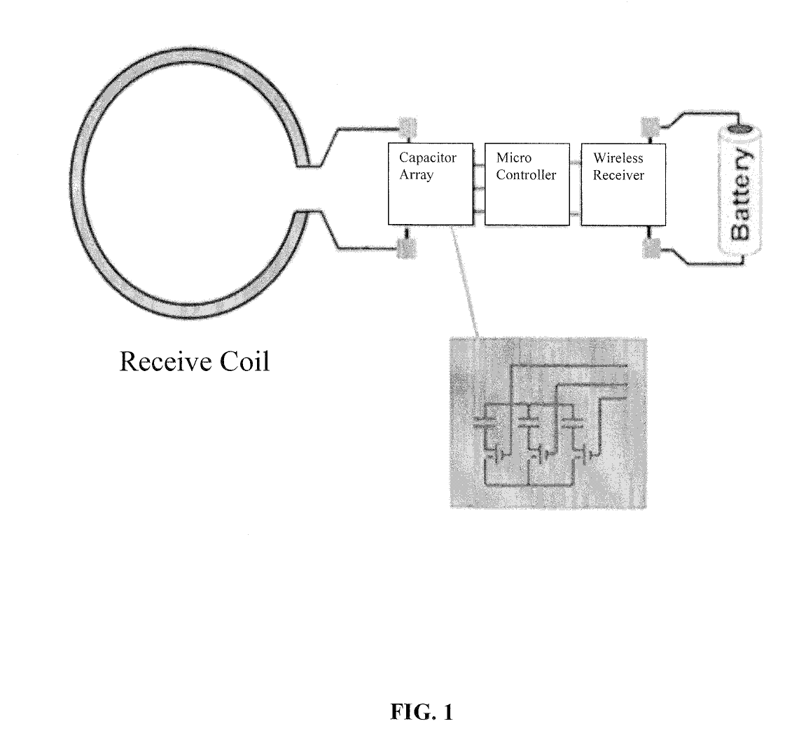

Embodiments of the invention relate to a method and apparatus for providing wirelessly-controlled multiple-frequency (MRI/S) coil system that can be implanted in a biological subject. Embodiments can also be utilized without implantation in a biological subject. Embodiments can address sensitivity limitations of current MRI/S technology and can be utilized for monitoring internal structures of biological systems. Embodiments can involve wireless control of a multi-frequency MRI/S coil system utilizing inductive coupling of a coil that can be implanted within the body of a biological system (in vivo) to one external coil. Inductive coupling is an effective method that can be used to increase the sensitivity of the MR scan. The detection of multiple important biological nuclei, such as ¹H, ¹⁹F, ³¹P, and ¹³C, is beneficial for a complete characterization of the biological system's function. Multiple-frequency designs presented in the literature have yielded low quality factor (Q) and signal-to-noise Ratio (SNR) because the generation of multiple frequencies requires extra components or generates unwanted modes which add loss to the coil system. To overcome this issue, embodiments of the invention can utilize an efficient varactor array and microcontroller to create a single resonance at any desired MRI/S frequency, with no extra modes generated. This allows a dynamic wireless selection of the coil's resonant frequency while maintaining high sensitivity. FIG. 1 shows a simplified block diagram of the implantable coil connected to a capacitor/varactor array, driven by a microcontroller that receives information wirelessly.

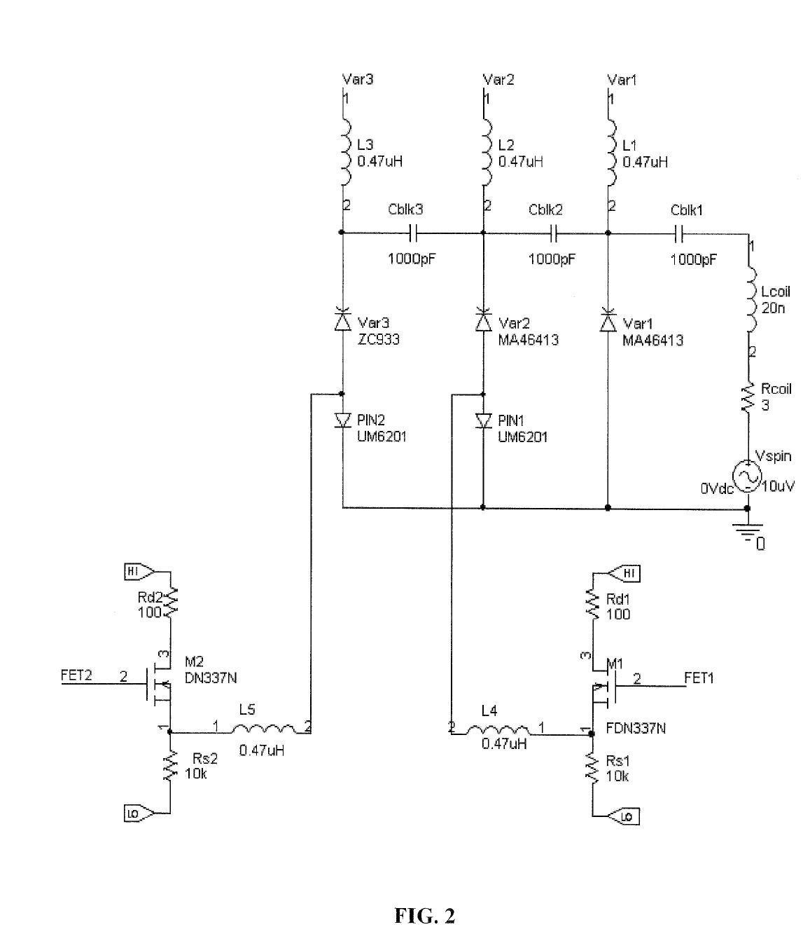

The switchable varactor/capacitor array, shown in FIG. 2, includes multiple parallel branches, each containing a varactor for tuning of the MRI/S coil. Each branch can be enabled via a PIN diode switch controlled by a FET. Any number of branches may be added to alter the tuning range. Another embodiment utilizes a CMOS switch as a replacement of the PIN diode and FET combination.

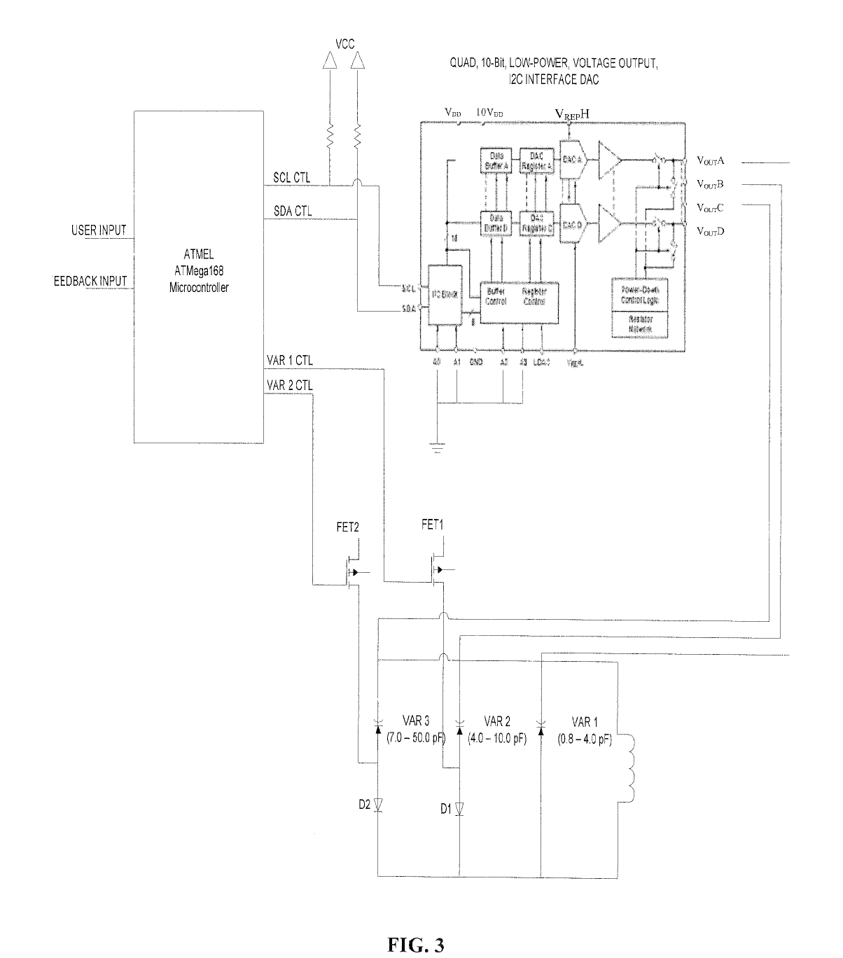

An embodiment of the digital design, shown in FIG. 3, includes three main functional components: (1) input to the microcontroller; (2) automated control of varactors via digital to analog converter (DAC); (3) automated control of FETs. The microcontroller analyzes the input information supplied by the user to select the operating MR frequency of interest. The controller determines the appropriate digital output to be sent to the FETs and DAC. The DAC converts this digital signal to an analog voltage used to control the varactors in the array.

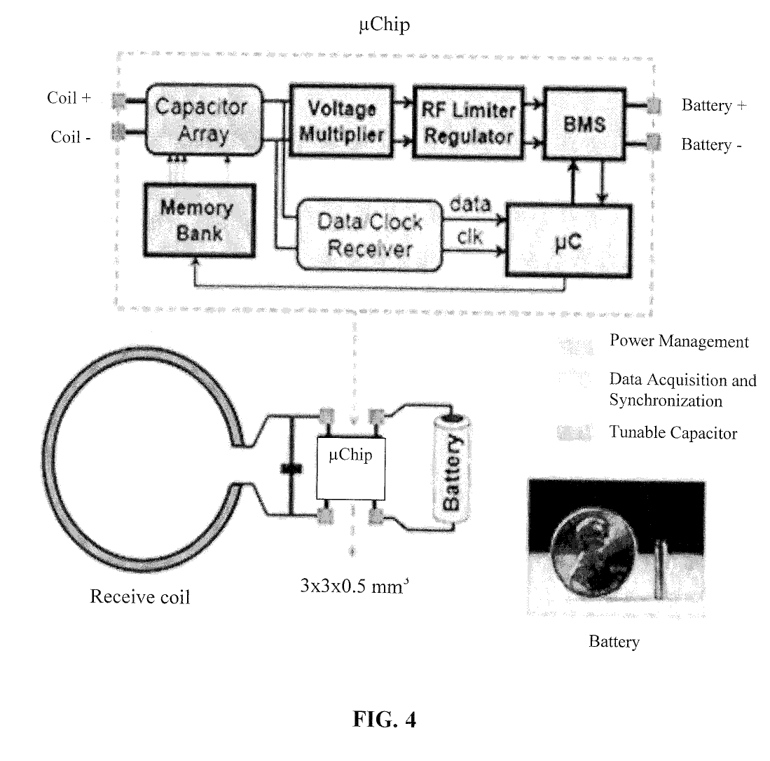

The microchip also includes an input scheme to accept and decode information from the user. Shown in FIG. 4 is a detailed breakout of the integrated microchip (measuring 3×3×0.5 mm³). The breakout shows the varactor/capacitor array, the micro-controller, as well as details involving the input information processing.

Embodiments of the invention can allow tuning to two or more of many nuclear magnetic resonances within a living system, at any MRI/S field strength. A specific embodiment allows tuning to any nuclear magnetic resonance within a living system, at any MRI/S field strength. Applications include, but are not limited to, implantable coils. Implantable coils can significantly increase the sensitivity of the MR scan. Non-implantable coils can be used, for example, in applications where such high sensitivity requirements do not exist, but where multiple-frequency requirements do exist, or adjustments to impedance matching under extreme loading.

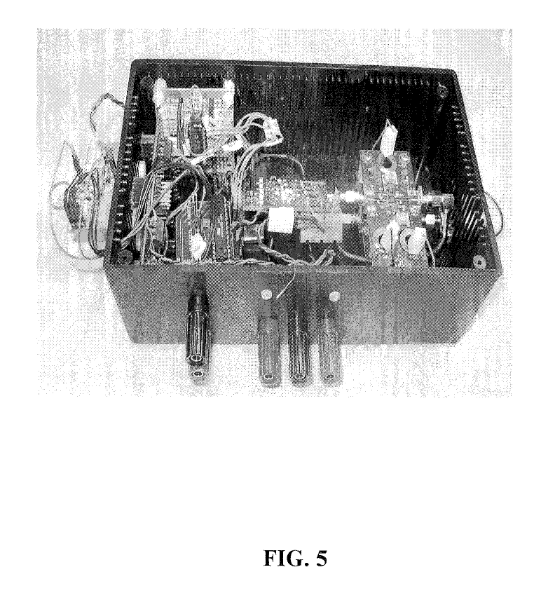

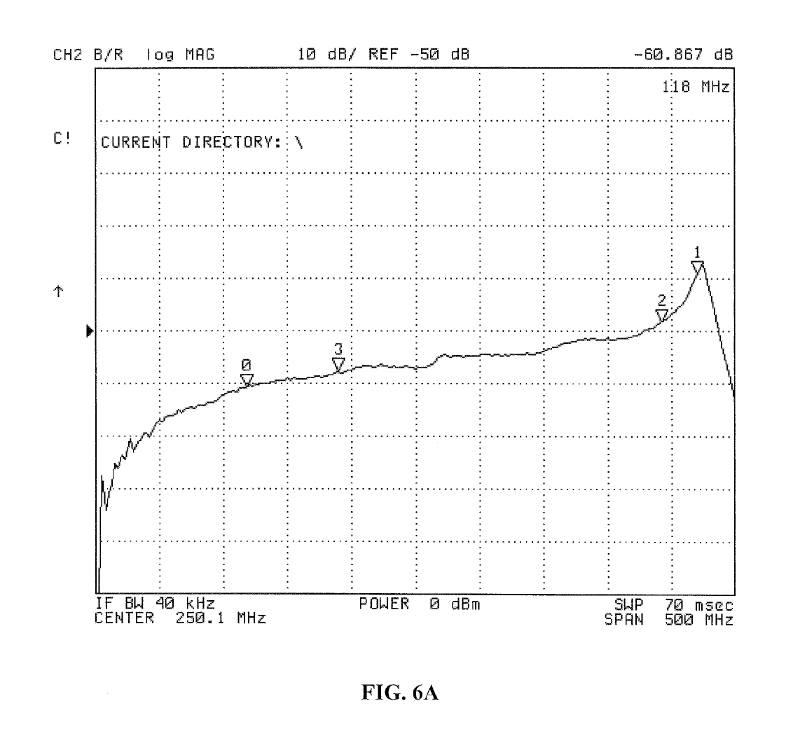

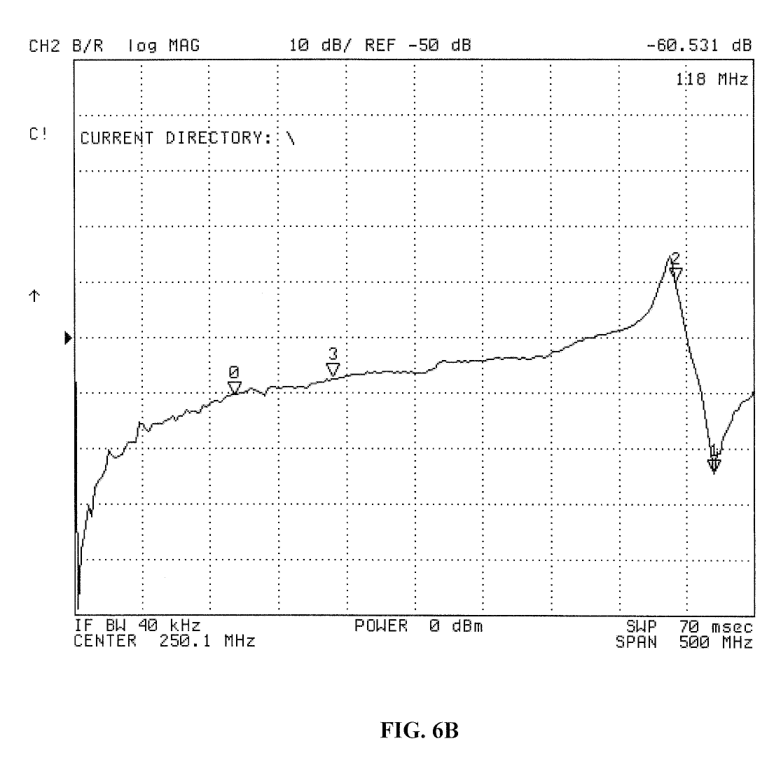

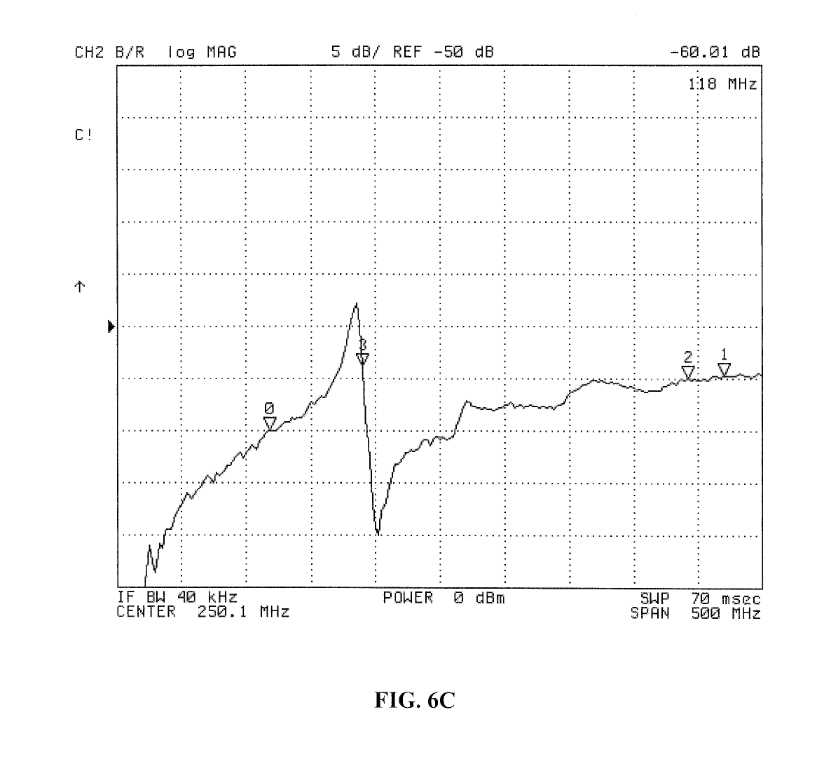

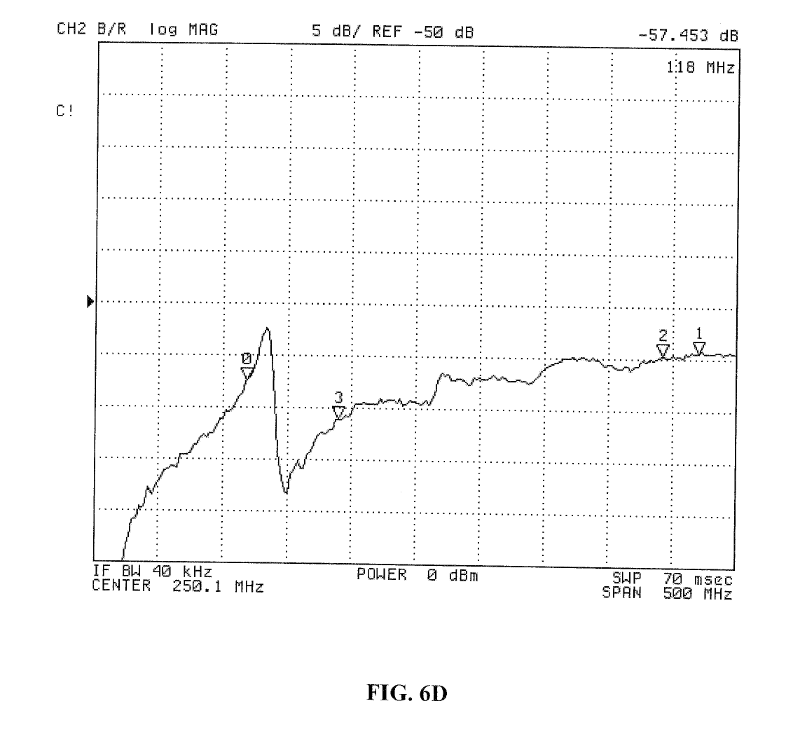

An embodiment of a microcontroller-driven varactor array that was designed, constructed, and tested, is shown in FIG. 5. The embodiment shown in FIG. 5 provides the ability to tune to multiple frequencies under wireless control. The results of dynamic switching of the embodiment of FIG. 5 to four selected frequencies are shown in FIG. 6. The network analyzer measurements of FIG. 6 confirm the capability of an embodiment to wirelessly tune to the individual frequencies of the four biological nuclei at 11.1T: 1H (470 MHz), 19F (442 MHz), 31P (190 MHz), and 13C (118 MHz). Additional embodiments can be microfabricated to reduce the size.

Embodiments of the remotely-tuned, multiple frequency implantable coil system can allow monitoring of the function of a construct, such as a tissue engineered pancreatic substitute, and correlation to the post-implantation physiological effects. The monitoring of a bioartificial pancreas is one example of a medical need that can be met by embodiments of the invention. Further, embodiments can be used to monitor the function and viability of other bioartificial organs, such as bioartificial kidneys, livers, and lungs. Other applications include monitoring of any tissue in the body for which MRI/S imaging signals can be beneficial and an implantable structure can be positioned proximate the tissue.

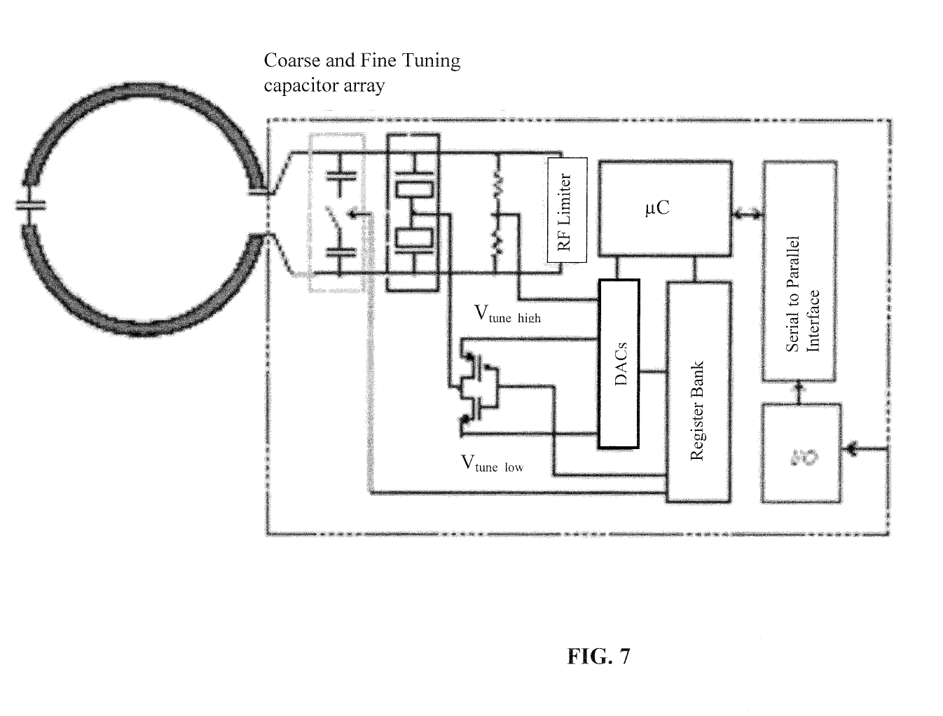

An embodiment of the invention, shown in FIG. 7, incorporates a mixed-signal integrated circuit fabricated in standard CMOS technology to selectively tune an implantable coil to the multiple MRI/S resonances. The integrated circuit includes a microcontroller, a register bank, a serial interface, and a digitally controlled capacitor array to tune the coil. The capacitor array has both coarse and fine tuning elements. The capacitors can be implemented in various forms available to standard CMOS process manufactures, for example, metal-insulator-metal (MiM), nMOS type or pMOS type active MOS capacitors. The capacitive elements can be connected in differential or single ended fashion. For differential connections, two capacitors are connected differentially with a series switch. This configuration allows the coil to be isolated from the supply network while facilitating biasing of the capacitor plates. This arrangement also eliminates the need for a bulky external isolation RF choke. Additional fine frequency tuning can be accomplished with a bank of digitally controlled varactors. The command words can be derived from a register bank to control both coarse and fine capacitive tuning. The entire capacitor tuning bank can be placed across the implantable coil, which has capacitor breaks to decrease the peak voltage on the chip during NMR transmission. Additional capacitor breaks can be added as required to ensure peak transmission voltages are within the breakdown limits of the devices. In an embodiment, to further ensure device reliability, an RF limiter can be integrated into the chip, that the RF limiter provides low impedance path to the induced current.

Embodiments of the invention also relate to a method and apparatus for non-invasive monitoring of a tissue engineered construct. An embodiment is directed to a high sensitivity NMR, selective wirelessly-adjustable multiple-frequency probe (SWAMP) system using an implanted coil that can be used to non-invasively monitor the function in vivo of a tissue engineered construct, such as a pancreatic substitute. An embodiment can incorporate a frequency selection microchip system having a digital-controller and tunable capacitor array to selectively tune an implantable coil to the NMR resonances of ³¹P, ¹⁹F, and ¹H at 11.1 Tesla (190, 442, and 470 MHz). A primary-battery power management circuit can be used with the implanted microchip system. An external automatic impedance matching system having varactors, a digital-controller, voltage controlled oscillator, and directional coupler for precise impedance matching of the inductively coupled implanted RF coil and the external RF coil. The external impedance matching system can be powered by the NMR console or other power supply.

The implant coil and external coil, which are inductively coupled, can integrate with the digital frequency and impedance selection system and the coil inductors in order to provide selective wirelessly adjustable multiple-frequency probe (SWAMP) operation. The implantable coil can be tuned through the inductively coupled wireless-interface to provide real-time, digital adjustment of the microchip on the implanted coil, and the external coil can then be used to impedance match the coupled coil system automatically. Standard console-controlled radio-frequency pulse sequence tools can be used to generate the coding sequence for remote programming of inductively coupled coils. In this manner, the coils can be selectively tuned to a desired resonance frequency and impedance matched, while superior NMR signal sensitivity is obtained in vivo using inductive coupling between the implanted and external coils.

Embodiments of the subject system can be compatible with existing MR instruments without modifications, where standard radio-frequency pulse sequence tools can be used to generate coding sequences to remote program the microchip system through the inductively coupled coils. An array of varactors and capacitors can be remotely switched, via a digital controller embedded within a microchip, to resonate with the inherent inductance of the coil. In this fashion, a short pulse sequence can be executed to switch the coils to the desired frequency and tune the coil impedance without moving the subject or changing any hardware. With this approach, the user can set the system to any desired frequency by communicating with this microcontroller. This coil then essentially behaves as a single-frequency resonant coil, significantly improving the SNR. Thus, by wireless adjustment of the resonance frequency in the very confined space of implantable coil, tuning and matching the external coil inductively-coupled to the implantable coil, and digitally controlling the selection of resonance frequency, for example, specific nucleus, a tissue construct can be monitored by measuring NMR images and spectra of nuclei from important metabolites in a single measurement session in the magnet.

Embodiments can enable the monitoring of tissue-engineered construct properties such as vascular permeability, oxygenation, metabolism, and pathophysiological changes, in vivo. Embodiments can be applied to various areas of tissue engineering, including: cardiovascular substitutes, such as blood vessels and heart values; orthopedic replacements, such as bone and cartilage; nervous tissue transplants, such as spinal cord; and the encapsulated cell therapies, including bioartifical constructs. Additional embodiments can be used to monitor constructs such as therapies to mimic salivary glands, endocrine tissues, such as hypothalamus, thyroid, adrenals, and the bioartificial pancreas. The subject NMR coil system can assess intra-construct metabolic activity by monitoring pO₂, ATP (as an index of cell bioenergetics) and TCho (as an index of cell viability), where changes in these metabolic indices may precede implant failure and end-point physiologic effect, such as hyperglycemia. The subject coil technology can enable prediction of implant failure while the recipient is still euglycemic.

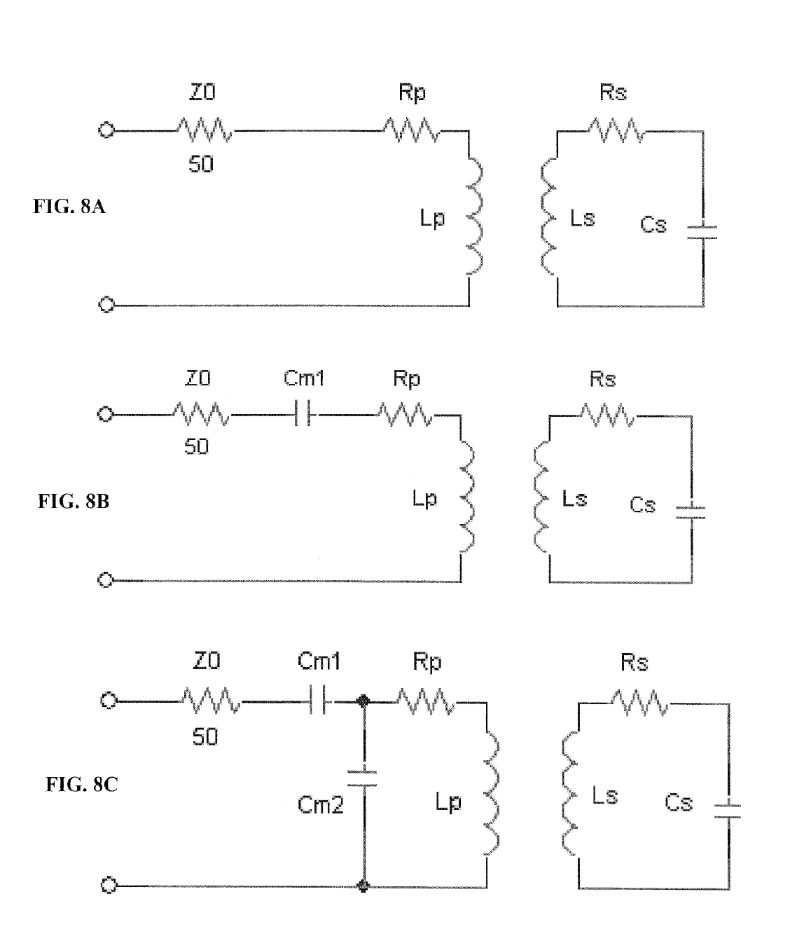

A configuration of inductively coupled coils is shown in FIG. 8A, in which the internal coil is resonant and the external coil is not resonant. With this configuration, impedance matching is achieved by adjusting the distance between the primary and secondary until the reactance coupled into the secondary is exactly cancelled by the reactance of the primary so that the impedance reaches the desired value. If the coil loading changes, the distance between the coils can be adjusted to rematch the impedance. An alternative embodiment is shown in FIG. 8B, where the distance between the loops remains constant and the reactance coupled into the primary is canceled with a capacitor in series with the primary inductor. In addition, the size of the primary inductor may be changed to alter the coupling between the primary and secondary. A third embodiment is shown in FIG. 8C, where a shunt capacitor has been added to the primary inductor. If the capacitor and primary inductor are chosen to create a resonance near the resonance of the secondary, the inductors are over-coupled and two modes are excited; a low-frequency mode in which the currents are in the same direction (co-rotating), and a high-frequency in which the currents are in opposite directions (counter-rotating). Impedance is matched with the combination of the shunt and series capacitors.

To understand the spatial distribution of the near-magnetic-fields from each coil configuration in FIGS. 8B-8C, circuits simulations were performed using GNEC (Nittany Scientific, Riverton, Utah). A resistor was added in the coil loops to emulate the sample induced resistive losses, because GNEC does not allow the specification of the surrounding load. In addition, the simulated coil structures were small compared to the wavelength of interest and thus the B1 field within the sample was not subject to the severe wave effects seen in large size high-frequency structures. The geometry of the coupled coil system included a larger coil (primary or external coil) separated by a distance of 1 cm from a smaller coil (secondary or implanted coil). Capacitors were added to the loops according to the circuits of FIGS. 8B and 8C, so that the coil systems were impedance matched. The results indicate that the configuration with the series tuned primary has a stronger magnetic field magnitude (11 A/m) at the location of the secondary coil in these simulation than the near-resonant primary (8.8 A/m), suggesting that the series tuned primary in FIG. 8B is preferable.

The simulations show that a series-tuned primary coupled to a parallel resonant secondary maximizes the signal strength of the inductively-coupled system. Single-tuned implantable coils significantly improve the signal reception from internal structures. This advantage can be extended to multiple nuclei by incorporating an automatic tuning mechanism into the implantable coil.

### EXAMPLE 1

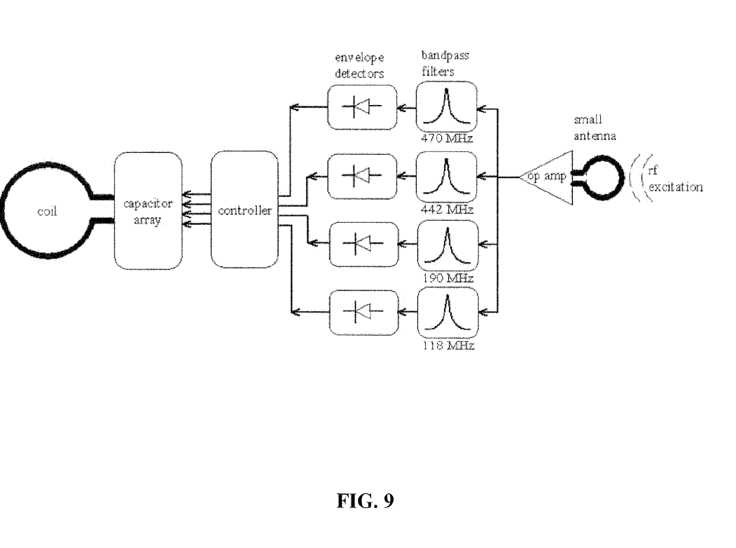

A system level block diagram of an embodiment of the subject device is shown in FIG. 9. The MR coil is directly connected to a capacitor array, which determines the MR coil frequency. The supporting circuitry includes a controller (ATmega168) to control the array and a wireless receiver, incorporating a small antenna, bandpass filters, and envelope detectors, to detect as input the user's desired frequency of operation. The overall digital system level design includes 3 main functional components: (1) buffering and amplification of filter input to the microcontroller; (2) automated control of varactors, via DAC converters; (3) automated control of the field-effect-transistor (FET) switches. Based on the input selected, the controller generates 2 outputs: (1) the appropriate data stream to a multiple-output DAC to generate an analog voltage for the varactors, and (2) a digital voltage for FETs to select the appropriate array branch to be activated. To select an MR frequency, the user simply sends an RF signal at the MR frequency of interest, which is detected by the small antenna and input in to the device circuitry.

The capacitor array (shown in FIG. 2) has three parallel branches, each containing a varactor for tuning of the NMR coil. The first branch (Var1) has only a varactor. The second and third branches (Var2 and Var3) have a varactor and PIN diode switch controlled by an FET. Varactor-1 and Varactor-2 (Macom model 46413) have capacitances between 0.8-4 pF for voltages ranging between 0-5 V. Varactor-3 (ZETEX model ZC933) has capacitances between 7-80 pF for voltages ranging between 0-5 V. The PIN diodes (UM6201) have an “on” resistance of 0.4 ohms and an “off” capacitance of 1.1 pF in parallel with 10k ohms. The transistors, M1 and M2, are N-channel enhancement mode field effect transistors (FDN337N) that provide the current necessary to forward bias the PIN's (˜40 mA) and the reverse bias voltage (˜−5V) necessary to turn off the PIN's. The 1000 pF capacitors are low loss ceramic chips with equivalent series resistance of 0.016 ohms. The 0.47 μH RF chokes are phenolic-core inductors with a parallel self-resonance frequency of 500 MHz.

Referring to the embodiment shown in FIG. 9, the Q of the entire capacitor array was measured by finding the −3 dB points (i.e. bandwidth, BW) for each frequency of interest, computing the Q (f₀/BW), and finally the equivalent-series-resistance (ESR) of the known coil inductor (ESR=ωL/Q). The −3 dB points were measured by loosely coupling to the MRI coil using two probes connected to the reflection and transmission ports of a vector network analyzer. The principle behind loose coupling is that one probe sources RF while the other senses RF. Any resonant circuit placed between the two probes will then absorb energy and this absorption response can be viewed on the transmission port of the analyzer. We were able to successfully switch the POC device frequency to each of the 4 frequencies. Measurements indicate that the ESR of the entire array is ˜1 ohm. The design was a success and demonstrated a flexible system that can be adapted to any specified NMR frequency. Additional embodiments of the system can include a microchip utilizing CMOS switches, which can allow removal of the PIN switches and large array circuit board of the POC device, to greatly reduce the surface area of the tuning circuit and reduce the losses suffered. Additional embodiments can remove the extra 3-cm antenna and the bandpass filters, and can use the NMR coil and pulse sequence program to efficiently input information to the microcontroller circuit.

### EXAMPLE 2

A selective wirelessly adjustable multiple-frequency probe (SWAMP) system can be used to tune and match inductively-coupled coils for excitation and detection of in vivo NMR from nuclei, ¹H, ³¹P and ¹⁹F, at 11.1 T.

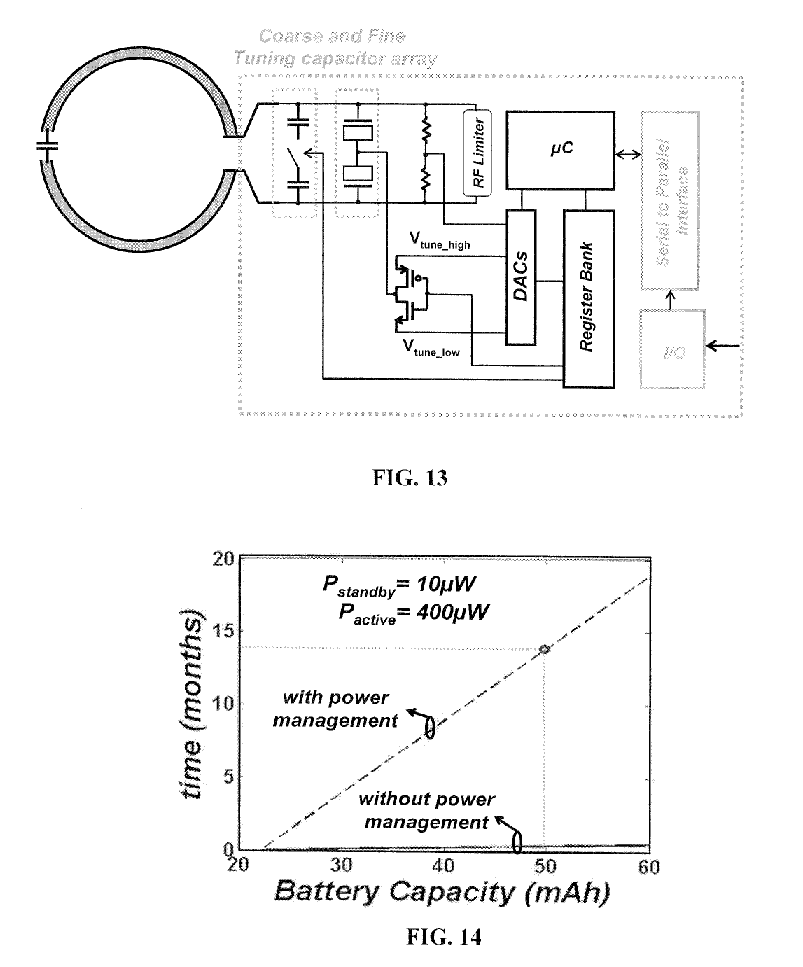

An integrated circuit (IC) can incorporate an implantable microchip fabricated in mainstream complementary-metal-oxide semiconductor (CMOS) technology that incorporates a digitally tunable capacitor array, a clock/data recovery receiver, a microcontroller with register bank and a power and battery management system. The microchip measures ˜3 mm×3 mm×0.5 mm and can be easily incorporated into the implant coil construct for wireless tuning in real-time to allow acquisition of NMR spectra at the desired frequencies. The overall architecture of the microchip in accordance with this embodiment is shown in FIG. 13 and includes three major functional blocks: (1) power management, (2) data acquisition and synchronization, and (3) tunable capacitor.

The embodiment utilizes a frequency selection microchip system having a digital-controller and tunable capacitor array to selectively tune an implantable coil to the NMR resonances of ³¹P, ¹⁹F, and ¹H at 11.1 Tesla (190, 442, and 470 MHz), a primary-battery power management circuitry for the implanted microchip system, and an external automatic impedance matching system containing varactors, a digital-controller, voltage controlled oscillator, and directional coupler for precise impedance matching of the inductively coupled implantable coil and external coil. The external impedance matching system can be powered by the NMR console.

The implantable circuit can be miniaturized onto a microchip and have an implanted coil surround the tissue construct. This circuit can be powered by a battery. The external circuit can automatically respond and adjust the match of the inductively-coupled coil system.

FIG. 13 shows an embodiment having a microcontroller, register bank, serial interface and digitally controlled capacitor array to tune the coil. The capacitor array has both coarse and fine tuning elements. Coarse capacitive tuning is provided by a bank of metal-insulator-metal (MiM) capacitors. For each bit, two capacitors are connected differentially with a series switch. The differential configuration allows the coil to be isolated from the supply network while facilitating biasing of the capacitor plates. This arrangement also eliminates the need for a bulky external isolation RF choke. In order to achieve fine frequency tuning, a bank of digitally controlled varactors can be implemented with nMOS transistors electrically connected, as illustrated in FIG. 13. The gate terminals of the varactors are connected to implant coil nodes and the source/drain terminals are connected to high and low tuning voltages via the switch network. A 24-bit digital word from a register bank can be used to control both coarse and fine capacitive tuning. The entire capacitor tuning bank is placed across the implantable coil, which has capacitor breaks to decrease the peak voltage on the chip during NMR transmission. Additional capacitor breaks can be added as required to ensure peak transmission voltages are within the breakdown limits of the devices. To further ensure device reliability, an RF limiter can be incorporated into the chip that provides low impedance path to the induced current.

The chip can incorporate a microcontroller, serial-to-parallel interface, and input/output circuitry to communicate with an external digital PC card. The custom controller has a low power 8-bit microprocessor with up to 16 read/write ports for flexible interfacing with internal mixed signal components. Control commands from the external card can be used to upload data into the register bank and tune the capacitors. The chip can also incorporate buffers and A/D circuitry to diagnose voltage level of internal references and determine the effect of the static magnetic fields and RF transmitter on the microchip performance.

Embodiments can incorporate a telemetry receiver and a wireless power interface with battery management system. ICs can be fabricated in 2-poly, 3-metal 0.6 μm CMOS process technology, and designed for inductively coupled coils. The telemetry chip can receive RF pulse sequences similar to those generated by an NMR console, acquiring data and clock signals using a modulation scheme based on amplitude shift keying (ASK) and pulse position modulation (PPM). FIGS. 10A and 10B show the measured voltages for the ASK demodulator circuit, along with recovered clock and data signals indicating correct reception of a “110” test pattern (the inset in FIG. 10A shows the receiver die photo). The receiver supports 4 kb/s to 18 kb/s, has a sensitivity of 3.2 mVpp, and a measured power dissipation of 70 μW at 2.7 V. Since higher voltages can be induced across the implant coil using the NMR console, the receiver sensitivity can be decreased, and the power dissipation in the system can be reduced by at least a factor of 50× by eliminating the front-end amplifier stage altogether.

The wireless power interface and battery management system chip can include a regulation and rectification circuit for extracting power from a wireless carrier, and a battery control loop for generating charging profiles and estimating the end-of-charge (EOC) of a secondary (rechargeable) battery. As shown in FIG. 10C, the measured transient regulator response is within 15% (or 600 mV/4.1V) of the target 4.1V supply, when an externally generated 0 to 2 mA load step is applied as the link is powered by the primary coil voltage. The regulator exhibits a load regulation of 2 mV/mA (or 240 ppm/1 mA), a line regulation 2 mV/V, and a low dropout voltage of 50 mV. The battery charger delivers 1.5 mA during the constant-current phase and produces the EOC signal during the constant-voltage phase once the battery current reaches 5% of the nominal charging current of 1.5 mA (see FIG. 10D). The measured power dissipation of the overall battery control loop is 160 μW, and the efficiency ranges from 66% to 95% depending on the charging phase. Since power is dissipated only when the battery is being charged and otherwise the control loop remains inactive, the actual power dissipation in standby mode is negligible and less than 1 μW. The inset in FIG. 10D shows the fabricated CMOS die attached onto a standard circular printed circuit board (PCB).

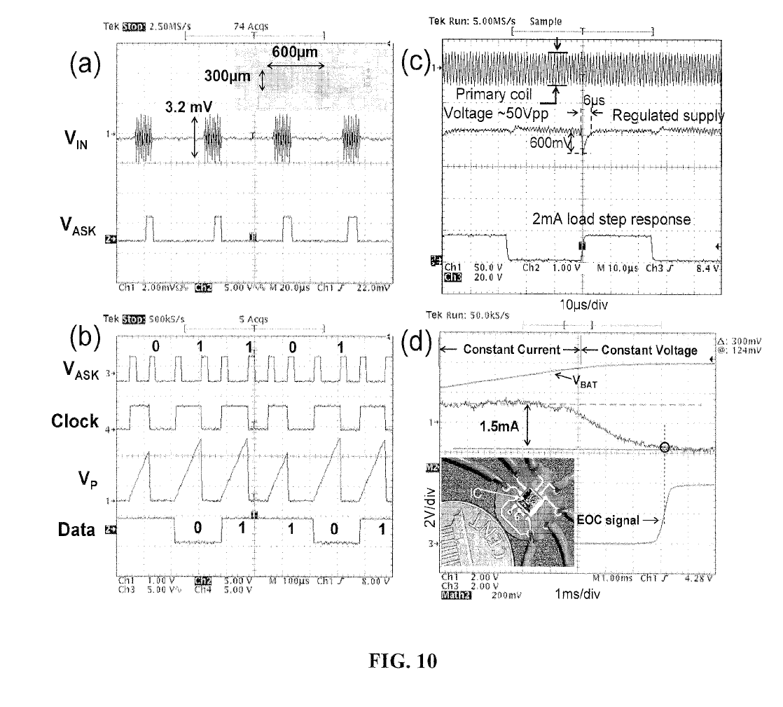

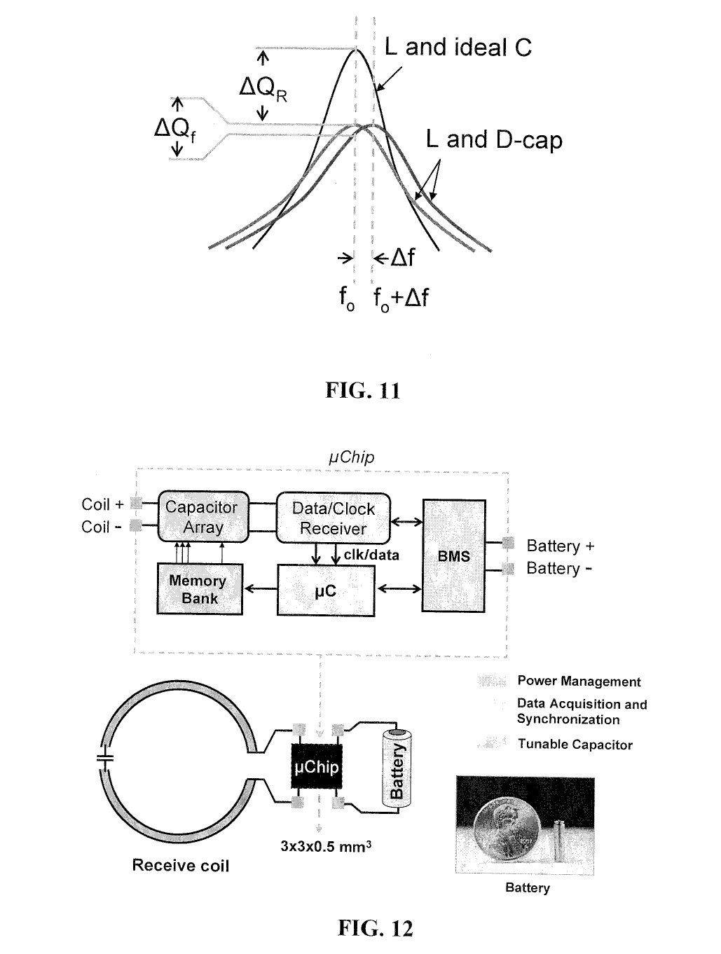

Two loss mechanisms arise from replacing a fixed value capacitor with a D-cap array as illustrated in FIG. 11. The first and most detrimental loss arises from the finite D-cap ESR, which is mainly determined by the resistance of digital switches. When a lossless (ideal) capacitor is replaced by the D-cap, the overall quality factor drops by ΔQ_{R}. The second loss mechanism, denoted ΔQ_{F}, is caused by the limited frequency resolution as a result of finite capacitance steps of the digital capacitor array. The total fractional loss in Q is ΔQ_{R}/Q+ΔQ_{f}/Q, where Q is the resonant-tank quality factor.

For a target fractional loss ΔQ_{R}/Q, the minimum acceptable resistance R_{c} of the capacitor and switch can be defined in terms of R_{L}, the tissue loaded coil ESR. For instance, a 10% fractional loss in Q requires an R_{c} less than R_{L}/9 for a D-cap quality factor Q_{Dcap} of ˜180 (this assumes a 20 nH coil with a measured Q of 20 in physiological equivalent gel at 470 MHz, the highest NMR frequency of interest). Since the on resistance Rₒₙ of a switch is inversely proportional to both the switch size and its parasitic capacitance Cₚ, the basic

TABLE 1
Estimated parameters for proposed D-Cap array.

| | | | | | Total % loss |
|---|---|---|---|---|---|
| ΔQ_{R}/Q | | R_{C} = KR_{L} | C_{PAR} | ΔQ_{F}/Q | ΔQ_{R}/Q + |
| (%) | Q_{Dcap} | (K) | (pF) | (%) | ΔQ_{F}/Q (%) |
| 10 | ~180 | 1/9 | 3.04 | 0.47 | 10.47 |
| 20 | ~80 | 1/4 | 1.35 | 0.36 | 20.36 |
| 30 | ~46 | 3/7 | 0.79 | 0.28 | 30.28 |

design challenge is to determine a sizing strategy for the D-cap array that yields a sufficiently low ESR to meet the desired Q constraints of the tank and also the smallest parasitic capacitance so as not to limit the highest NMR frequency of interest (e.g., 470 MHz in a specific situation). A sizing approach that maximizes the RC time constant, formed by the resonant capacitor and its loss resistance (R_{C}) at each of the desired NMR frequencies, may produce the most optimal results. This approach satisfies the Q requirements at each NMR frequency using the smallest possible switch size and hence the smallest parasitic capacitance. The “on” resistance Rₒₙ of a minimum-sized transistor in 130 nm standard CMOS process is ˜2.3 kO and the corresponding parasitic capacitance Cₚ at the drain node; ˜0.3 fF. A 10% degradation in overall Q yields a total parasitic capacitance of ˜3 pF, which is well below the 5.73 pF capacitance required to resonate a 20 nH loop at 470 MHz (Table 1). Moreover, the impact of fractional loss ΔQ_{F}/Q due to finite frequency stepping in the D-cap array appears to be negligible (Table 1). This assumes a minimum capacitance resolution or least significant bit (LSB) of 31.25 fF, which is well above the minimum capacitances that can be designed in a 130 nm process.

Power dissipation estimates for embodiments of the SWAMP microchip along with measured data for a specific device and IC implementations are shown in Table 2. The basic components of the SWAMP device are the receiver, controller, battery management circuit, digital capacitor (D-cap) array and the battery. If a 3-3.6 V Li-ion primary battery is used, a linear regulator will be required to supply the 1.2 V for the microchip electronics. A more advanced CMOS technology can be used and, hence, lower the supply voltage, as the devices and passive components exhibit lower loss and improved performance for the D-cap array implementation. In standby mode, the SWAMP microchip is estimated to consume less than 10 μW, whereas in active mode the overall current draw from a 3.6 V is about 100 μA.

TABLE 2
Power dissipation estimates and measured data for a specific embodiment.

| | Power Dissipation (A: Active, S: Standby) | | | |
|---|---|---|---|---|
| | | | Battery | |
| Design components | Receiver | Controller | Management | Capacitor Array |
| POC SWAMP | 90 mW | 1.3 mW | — | 200 mW |
| device (1.8-5 V) | | | | |
| Preliminary CMOS | 70 μW | — | 165 μW (A) | — |
| prototypes (2.7 V-3 V) | (A&S) | | <1 μW (S) | |
| SWAMP microchip | <1.5 μW¹ | <1 μW² (A) | <240 μW³ (A) | <120 μW (A) |
| (1.2 V) | (A&S) | <100 nW (S) | <7 μW (S) | <100 nW (S) |

Table 2 shows receiver sensitivity ˜500 mV (no amplification stage), 1.2 V supply, total receiver bias current of 1 μA, yields ˜1.20 μW. Assume gate cap of 2 fF/um, average gate width in standard cells ˜2 μm, controller with 10,000 transistors, 1.2 V supply, frequency 10 kHz, yields power dissipation ˜0.575 μW. Input battery voltage 3-3.6 V (Li-ion battery), output of linear regulator 1.2 V, load current ˜100 μA when active, yields (3.6 V-1.2 V)×100 μA ˜240 μW. In standby mode, load current is ˜1-2 μA, which yields power dissipation ˜3-7 μW. In active mode, estimated current draw is 100 μA which yields ˜120 μW. In standby, all D-cap array components are shut down dissipating negligible current.

The microchip can be powered by a primary Li-ion biocompatible pin-type battery. In other embodiments, a secondary (rechargeable) battery can be used. A Contego Series battery from EaglePicher Medical Power (Surrey, B.C. Canada), specifically designed for medical implants, that has a low magnetic signature (titanium enclosed) and is NMR compatible can be used. The battery measures 6.0 mm×12.0 mm×15.54 mm and is rated at 55 mAh with a peak discharge of 110 mAh.

The device can be used to non-invasively monitor the function in vivo of an implanted pancreatic substitute. For this task, the device should be operational for at least 6 months. Therefore, a battery management system (BMS) with fast entry and exit strategies from power down/active modes can be developed. FIG. 14 shows the required battery capacity and estimated device duration with and without a battery management system. A battery rated at 50 mAh operated for 200 hrs (equivalent to 50 NMR experiments, each 4 hrs in duration) can last up to 14 months—this assumes an active and standby power dissipation of 10 μW and 400 μW, respectively, at the operating cell voltage of 3.6 V.

For long-term in vivo characterization of engineered tissues, the battery management system can feature power gating transistors to disable the register bank, capacitor array, on-chip regulators, and non-critical diagnostic circuits. In an embodiment, the receiver and the microcontroller can be the only elements that remain active at all times. To minimize current consumption during “sleep/standby” mode, the gain and sensitivity of the receiver can be dynamically adjusted by decreasing the current bias of the amplification stages. The power dissipation of the microcontroller should be negligible (˜nW range), since the clock recovery module does not generate clock signals to gate the controller during standby mode.

Another chip can be fabricated in CMOS technology. The device can be packaged in low profile quad-flat package (LQFP) and wire-bonded using gold wires. The package measures 5 mm×5 mm and <2 mm in height and is soldered onto a copper printed circuit board and connected to a battery via twisted pair of cables. The receiver can be fabricated to communicate with the device, and the entire system can be encapsulated in PDMS.

The device can include an automatic impedance matching system for the external coil, which has a digital controller with tunable capacitors (varactors) and diode components, powered by the NMR instrument console.

Selective implanted coil tuning with the microchip provides high sensitivity at each of the NMR nuclei. This information is then inductively-coupled to the external coil and the whole system is impedance matched to the characteristic impedance of the NMR system (e.g., 50 O). Each time the internal coil frequency is changed, a different impedance is coupled to the external coil, which requires a change in the external coil impedance matching network. Therefore, the external coil can provide automatic impedance matching when the frequency of the implanted coil is changed.

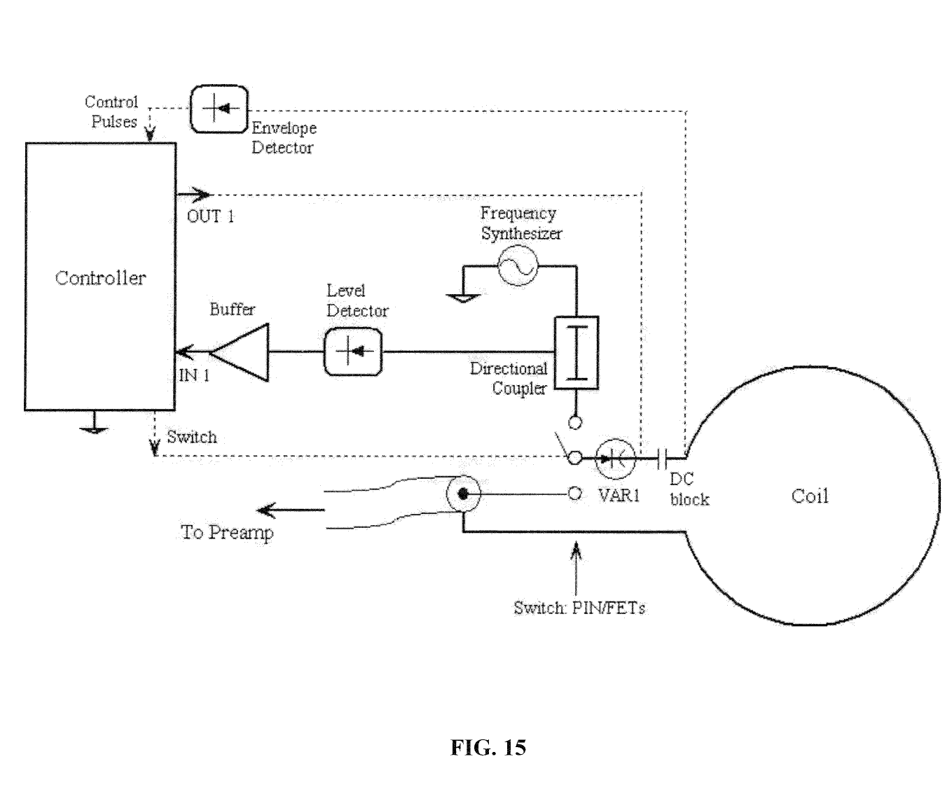

An automated impedance matching system can be used that takes advantage of similar technique used to tune the implantable coil. The control pulses sent from the MR console can be detected by the internal and external coil. The external coil can wait for the implanted coil to be tuned and then the automated impedance matching begins. A block diagram of this embodiment is shown in FIG. 15. When the controller receives the control pulses, the coil leads will be switched via PIN diode/FET switches, after a short delay, to the tuning circuit and provide a mid-range voltage to the varactor. The controller activates a programmable frequency synthesizer that outputs the appropriate frequency to a 50 O directional coupler and out to the coil. The reflected voltage will be detected, buffered, and input to the controller. The controller checks the level against a predefined value of minimal reflected power, resulting in a good impedance match. The controller can continue to vary the voltage applied to varactor until the level goes below the reference value. The process is complete when the detected signal from the varactor is below the reference. The controller then shuts down the frequency synthesizer, hold the varactor voltage, and switch the coil back to the system input.

The digital capacitor array can provide the necessary range and resolution for the NMR frequencies; the Q degradation is preferably within 10%-20%. The automatic impedance matching system should preferably generate a return loss =−20 dB, with −20 dB equal to 10% deviation from 50 Ω.

A specific embodiment of the microchip includes the capacitor array, a register bank and controller, a receiver, and the battery management system. The NMR-console-controlled RF pulse sequence can be used to upload digital words into the register bank to tune the capacitor array. Data transmission can be accomplished through inductive coupling between the external and implanted coil and received by an amplitude shift keying (ASK) receiver and clock/data recovery circuit. System data can also be uploaded to enforce the state for the controller, such as active mode, sleep mode, and programming internal elements. A cyclic redundancy checker (CRC) can be implemented for data integrity. The pulse sequencing can be organized into 64-bit data packets with proper header information to separate each packet. In the event of an error, the corresponding data packet is discarded until correct data is uploaded.

As the microchip interfaces to the external NMR controller via the same implantable coil used for NMR signal detection, an additional antenna is not required. In alternative embodiments, an additional antenna can be used. The receiver sensitivity of the microchip can be relaxed as the amplitude of the NMR-console generated RF pulses can be adjusted externally. Therefore, the receiver can detect pulses even if the implanted coil resonance is not matched to the RF pulses generated by the NMR console. Another advantage of this approach is that it enables the use of existing hardware and is therefore fully compatible with any NMR system. The software for data packet generation can simply be uploaded into the computer console of the NMR system. In addition, digital encoding of data packets and the CRC unit in the implant microchip does not allow the controller to inadvertently load incorrect data into the register bank during regular NMR measurements.

The chip can be packaged and mounted on a PCB with signal traces for the battery and the implantable coil. The implantable coil can be a single turn loop-gap circular inductor, having a 12 mm diameter, a 2 mm height, and constructed with 200 μm thick copper foil. This coil can have four distributed capacitors that minimize electric field losses to the sample and reduce voltages that appear at the terminals of the microchip. The system can be coated with PDMS.

The external coil can be interfaced to digital controller system, so as to provide NMR instrument power to the controller and optimize the inductive coupling between the external and implantable coated coil systems. The external coil can be directly driven during excitation and coupled to the implantable coil during excitation and reception. The external coil can be attached to the automatic impedance matching system and coaxial cable to provide NMR system connection. The mutual inductance between two single-turn parallel coaxial coils is determined by the radius of each coil and the distance between the coils. In a specific embodiment of the subject coil system, the radius of the internal coil (12 mm) and the distance between the two coils (˜15 mm) are determined by the anatomy of interest, which is the bioartificial pancreas implanted in a body. A variable left to adjust the mutual inductance is then the radius of the external coil. The impedance looking into the primary of the coupled coil system at resonance is Zᵢₙ=Qk²ω₀Lₚ, where Lₚ is the external coil, Q is the quality factor of the coil system, cop the resonant frequency and k=M/v(Lₚ Lₛ) is the coefficient of coupling. Therefore, the quality factor of the system can also be considered in designing the external loop. In addition, the external coil should preferably provide sufficient coupling across a wide frequency span. A 30-35 mm diameter surface coil can be sufficient to impedance match across all frequencies in accordance with an embodiment of the subject system.

All components of the automatic impedance matching circuit can preferably be located as close as possible to the coil input. Non-magnetic varactors are available in ranges required for impedance matching of the embodiments of SWAMP system. Depending on their magnetic properties, the other components of the automatic impedance matching circuit can be located as close as possible to the coil input. RG58 coaxial cable can be used to connect the external coil to the NMR system and cable traps can be positioned as needed. All components can be fixed to a planar fiberglass tray and sit in an available cradle.

The NMR system pulse programming capabilities can be used to program the internal and external coil controllers to the desired frequency and impedance using sequence of low power radio-frequency pulses.

The NMR-console can generate a sequence of pulses to communicate with the implanted SWAMP microchip. The pulse sequence can be programmed using standard Bruker pulse programming tools in the usual manner for any NMR pulse sequence on the 11.1 T Avance console. The RF pulse sequence can be used in a signaling scheme based on both amplitude shift keying (ASK) and pulse position modulation (PPM) to set the SWAMP system to the desired frequency (nucleus). The pulse sequence encodes every bit of information into three RF pulses. The first and last pulses define the duration of each bit (or the bit time T_{B}) and are used to facilitate clock signal recovery and synchronize the microchip to the external console. The relative timing position of the second RF pulse defines a “1” bit (logical high) or a “0” bit (logical low). Specifically, a logical “1” is encoded when the time between the first and second pulse is 60% of T_{B} and a logical “0” is encoded when the time between the first and second pulse is 40% of T_{B}. In this manner, a data packet of encoded ones and zeroes can be generated by the NMR system.

A protocol for operating a specific embodiment of the subject SWAMP system can be the following: First, execute the SWAMP system pulse sequence to select the frequency (nucleus) of interest. Then switch the NMR instrument to the appropriate frequency and perform the desired NMR measurements for the nucleus of interest. Once this is complete, execute the SWAMP system pulse sequence again to select the next frequency (nucleus) of interest. Then switch the NMR instrument frequency and perform the next NMR measurements. This process can be continued until all the nuclei and type of measurements have been completed. With modern NMR instruments (like the Bruker Avance system) and the SWAMP system, this process can be fully automated.

Within the SWAMP system, the automatic impedance matching system should preferably generate a return loss =−20 dB at each NMR frequency, with −20 dB equal to 10% deviation from 50Ω. The SNR of the SWAMP system should preferably be within 15% of the SNR of a single loop coil at each frequency.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for magnetic resonance imaging, comprising:
implanting an implant structure into a body, wherein the implant structure comprises:
a RF coil, wherein the RF coil detects changing magnetic fields and produces and output RF signal;
a mechanism for adjusting an impedance of the RF coil so as to select a resonance frequency of the RF coil, wherein the mechanism for adjusting the impedance of the RF coil is capable of receiving input regarding a desired resonance frequency;
locating an external RF coil external to the body, wherein the external RF coil is inductively coupled to the RF coil;
exciting a portion of the body proximate the implant structure with RF excitation;
detecting the output RF signal produced by the RF coil and inductively coupled to the external RF coil.

2. The method according to claim 1, wherein the implant structure is compatible with implantation in a living body.

3. The method according to claim 1, wherein the mechanism for adjusting the impedance of the RF coil allows the RF coil to have a resonance frequency at each of at least two frequencies.

4. The method according to claim 3, wherein the at least two frequencies correspond to at least two biological nuclei.

5. The method according to claim 1, wherein the mechanism for adjusting the impedance of the RF coil allows the RF receiving coil to have a resonance frequency at each of at least three frequencies.

6. The method according to claim 5, wherein the at least three frequencies correspond to at least three biological nuclei.

7. The method according to claim 4, wherein the at least two biological nuclei comprise two of the following ¹H, ¹⁹F, and ³¹P.

8. The method according to claim 6, wherein the at least three biological nuclei comprise ¹H, ¹⁹F, and ³¹P.

9. The method according to claim 1, wherein the mechanism for adjusting the impedance of the RF coil further comprises a microcontroller, wherein the microcontroller is capable of receiving wireless communication providing the desired resonance frequency, wherein the microcontroller controls the mechanism for adjusting the impedance of the RF coil to adjust the impedance of the RF receiving coil.

10. The method according to claim 1, wherein the mechanism for adjusting the impedance of the RF coil comprises a varactor array.

11. The method according to claim 1, wherein the mechanism for adjusting the impedance of the RF coil comprises a capacitor array.

12. The method according to claim 7, wherein the RF coil receives the wireless communication providing the desired resonance frequency.

13. The method according to claim 1, further comprising:
impedance matching the external RF coil after the implant structure is implanted in the body.

14. The method according to claim 1, further comprising:
adjusting the impedance of the RF coil to select at least two resonant frequencies.

15. The method according to claim 14, further comprising:
impedance matching the external RF coil after selection of each of the at least two resonant frequencies.

16. The method according to claim 13, wherein impedance matching the external RF coil is accomplished via an external impedance matching system, wherein the external impedance matching system comprises:
a plurality of varactors.

17. The method according to claim 16, wherein the external impedance matching system further comprises:
a digital controller;
a voltage controlled oscillator; and
a directional coupler.

18. The method according to claim 1, wherein exiting the portion of the body proximate the implant structure with RF excitation comprises exiting the portion of the body via the RF coil.

19. The method according to claim 18, wherein the RF excitation is coupled to the RF coil from the external RF coil.

20. The method according to claim 15, wherein impedance matching the external RF coil is accomplished via an automatic impedance matching system.

21. The method according to claim 9, wherein the microcontroller is capable of receiving wireless communication from pulse sequences from an MRI scanner.

22. The method according to claim 1, wherein the RF coil is wireless.

23. An apparatus for magnetic resonance imaging, comprising:
an implant structure, wherein the implant structure comprises:
a RF coil, wherein, upon exciting a portion of the body proximate the implant structure, the RF coil detects changing magnetic fields and produces and output RF signal;
a mechanism for adjusting an impedance of the RF coil so as to select a resonance frequency of the RF coil, wherein the mechanism for adjusting the impedance of the RF coil is capable of receiving input regarding a desired resonance frequency;
an external RF coil, wherein the external RF coil is inductively coupled to the RF coil;
a detector, wherein the detector detects the output RF signal produced by the RF coil and inductively coupled to the external RF coil.

24. The apparatus according to claim 23, wherein the implant structure is compatible with implantation in a living body.

25. The apparatus according to claim 23, wherein the mechanism for adjusting the impedance of the RF coil allows the RF coil to have a resonance frequency at each of at least two frequencies.

26. The apparatus according to claim 25, wherein the at least two frequencies correspond to at least two biological nuclei.

27. The apparatus according to claim 23, wherein the mechanism for adjusting the impedance of the RF coil allows the RF receiving coil to have a resonance frequency at each of at least three frequencies.

28. The apparatus according to claim 27, wherein the at least three frequencies correspond to at least three biological nuclei.

29. The apparatus according to claim 26, wherein the at least two biological nuclei comprise two of the following ¹H, ¹⁹F, and ³¹P.

30. The apparatus according to claim 28, wherein the at least three biological nuclei comprise ¹H, ¹⁹F, and ³¹P.

31. The apparatus according to claim 23, wherein the mechanism for adjusting the impedance of the RF coil further comprises a microcontroller, wherein the microcontroller is capable of receiving wireless communication providing the desired resonance frequency, wherein the microcontroller controls the mechanism for adjusting the impedance of the RF coil to adjust the impedance of the RF receiving coil.

32. The apparatus according to claim 1, wherein the mechanism for adjusting the impedance of the RF coil comprises a varactor array.

33. The apparatus according to claim 1, wherein the mechanism for adjusting the impedance of the RF coil comprises a capacitor array.

34. The apparatus according to claim 29, wherein the RF coil receives the wireless communication providing the desired resonance frequency.

35. The apparatus according to claim 23, further comprising:
a means for impedance matching the external RF coil after the implant structure is implanted in the body.

36. The apparatus according to claim 23,
wherein the mechanism for adjusting the impedance of the RF coil allows adjusting the impedance of the RF coil to select at least two resonant frequencies.

37. The apparatus according to claim 36, further comprising:
a means for impedance matching the external RF coil after selection of each of the at least two resonant frequencies.

38. The apparatus according to claim 35, wherein the means for impedance matching the external RF coil comprises an external impedance matching system, wherein the external impedance matching system comprises:
a plurality of varactors.

39. The apparatus according to claim 38, wherein the external impedance matching system further comprises:
a digital controller;
a voltage controlled oscillator; and
a directional coupler.

40. The apparatus according to claim 23, further comprising a means for exiting the portion of the body proximate the implant structure with RF excitation via the RF coil.

41. The apparatus according to claim 40, wherein the RF excitation is coupled to the RF coil from the external RF coil.

42. The apparatus according to claim 37, wherein the means for impedance matching the external RF coil comprises an automatic impedance matching system.

43. The apparatus according to claim 31, wherein the microcontroller is capable of receiving wireless communication from pulse sequences from an MRI scanner.

44. The apparatus according to claim 23, wherein the RF coil is wireless.
